(19) United States
(12) Patent Application Publication
(10) Pub. No.: US 2014/0134561 A1
(43) Pub. Date: May 15, 2014

(54) Point-of-Care System

(71) Applicant: Integrated Bionics, LLC, Richmond, TX (US)

(72) Inventors: Stéphane Louis Smith, Richmond, TX (US); Yves Kevin Smith, Richmond, TX (US)

(21) Appl. No.: 14/076,849

(22) Filed: Nov. 11, 2013

Related U.S. Application Data
(60) Provisional application No. 61/724,991, filed on Nov. 11, 2012.

(51) Int. Cl. A61B 5/00; A61F 5/56; A61C 7/08; A61C 13/08; A61C 19/04; A61B 5/1455; A61B 5/1468; A61B 5/0205; A61C 5/08; A61C 5/00; A63B 71/08; A61B 5/11

(52) U.S. Cl. CPC A61B 5/682; A61B 5/002; A63B 71/085; A61F 5/566; A61C 7/08; A61C 13/08; A61B 5/1118; A61B 5/14551; A61B 5/1468; A61B 5/0205; A61C 5/08; A61C 5/007; A61B 5/4833; A61C 19/04; A61B 5/053
USPC 433/6; 600/300; 433/215; 433/167; 600/595; 600/324; 600/547; 600/345; 600/531; 600/301; 433/218; 433/27

(57) ABSTRACT

The present embodiments relate to health monitoring systems and, in certain embodiments, to take-home monitoring systems designed for oral appliance monitoring. Embodiments include a method for obtaining data from an oral appliance. The method comprises placing an oral appliance comprising embedded electronics in an oral cavity, wherein the embedded electronics collect data; transmitting the data to a reader, wherein the reader comprises a computer, tablet computer, smart device, reader box, relay transmitter, or a combination thereof.

### CROSS REFERENCE TO RELATED APPLICATIONS

This application is a non-provisional of U.S. Application Ser. No. 61/724,991 filed Nov. 11, 2012, which is herein incorporated by reference in its entirety.

### BACKGROUND

Removable oral appliances are widely used to treat chronic conditions such as missing teeth, bruxism, and obstructive sleep apnea (OSA). Additionally, they may be used to prevent acute dental trauma resulting from traumatic head impact or direct tooth impact during athletic or military use. Typically a clinician makes a dental impression or uses a formable, thermoplastic material to fabricate a removable oral appliance. Following the fitting of the appliance, the patient may return to the clinic for regularly scheduled follow-up visits to monitor treatment efficacy.

In this traditional model, the patient may visit with a practitioner at regular fixed intervals. For each visit, the clinician may have the opportunity to service the oral appliance, obtain usage feedback, compliance measurements, and study the overall effectiveness of the treatment. The clinician may then choose to act upon that feedback such as correcting errors or making adjustments. There may be a trade-off between the frequency of visits and the delay in which the practitioner has an opportunity to act upon the feedback. Should the treatment require adjustment, increasing visit frequency may allow the practitioner to act quickly and intervene sooner potentially resulting in a better and more responsive treatment. However, increasing visit frequency may also increase the cost of using oral appliances for the patient, practitioner, and the insurance provider.

Checkups and appliance adjustments may be needed at a much higher frequency than what is practical. There are many cases where it may be desirable to increase the frequency of checkups and adjustments. Examples include, but are not limited to, prescription parameters that change faster than a fixed interval model can adapt to, the use of systems that require continuous monitoring (e.g., compliance monitoring), critical event notification such as a broken appliance, or electronic or mechatronic feedback systems embedded in an oral appliance.

### SUMMARY

The present embodiments relate to health monitoring systems and, in certain embodiments, to take-home monitoring systems designed for oral appliance monitoring. Embodiments comprise a reader. A reader may comprise a computer, tablet, smart device, reader box, and the like.

An embodiment may comprise a method for obtaining data from an oral appliance, the method comprising: placing an oral appliance comprising embedded electronics in an oral cavity, wherein the embedded electronics collect data; transmitting the data to a reader, wherein the reader comprises a computer, tablet, smart device, reader box, relay transmitter, or a combination thereof.

Another embodiment may comprise a system for obtaining data from an oral appliance, the system comprising: an oral appliance; a reader; wherein the reader is configured to communicate with the oral appliance, and wherein the reader is configured to download data from the oral appliance; a remote computer, wherein the remote computer is configured to receive transmitted data from the reader, and wherein the reader and the remote computer are configured to bi-directionally communicate; and a portal, wherein the portal is configured to display the data transmitted to the remote computer.

The features and advantages of the present invention will be readily apparent to those skilled in the art. While numerous changes may be made by those skilled in the art, such changes are within the spirit of the invention.

### BRIEF DESCRIPTION OF THE DRAWINGS

The following figures are part of the present specification, included to demonstrate certain aspects of embodiments of the present disclosure and referenced in the detailed description herein. Unless otherwise noted, figures are not drawn to scale.

FIG. 1 is an illustration of an embodiment from an exploded view showing the oral appliance and Reader components as they may be combined in an application.

FIG. 2A illustrates the top-down view of an embodiment of the top circuit board implementation for an oral appliance-reader communication module.

FIG. 2B illustrates the top-down view of an embodiment of the bottom circuit board implementation for an oral appliance-reader communication module.

FIG. 3 illustrates a flow chart depicting a Point-of-Care system where communication between an oral appliance and Reader occurs when the oral appliance is not in use, and is thus stored in the reader's compartment.

FIG. 4 illustrates a flow chart depicting a Point-of-Care system where communication between an oral appliance and Reader occurs when the oral appliance is actively in use.

### DETAILED DESCRIPTION

The present embodiments relate to health monitoring systems and, in certain embodiments, to take-home monitoring systems designed for oral appliance monitoring. Embodiments comprise a reader which may bi-directionally communicate with an oral appliance to read and transmit data. A reader may comprise a computer, tablet computer (referred to herein as a “tablet”), smart device, reader box, and the like.

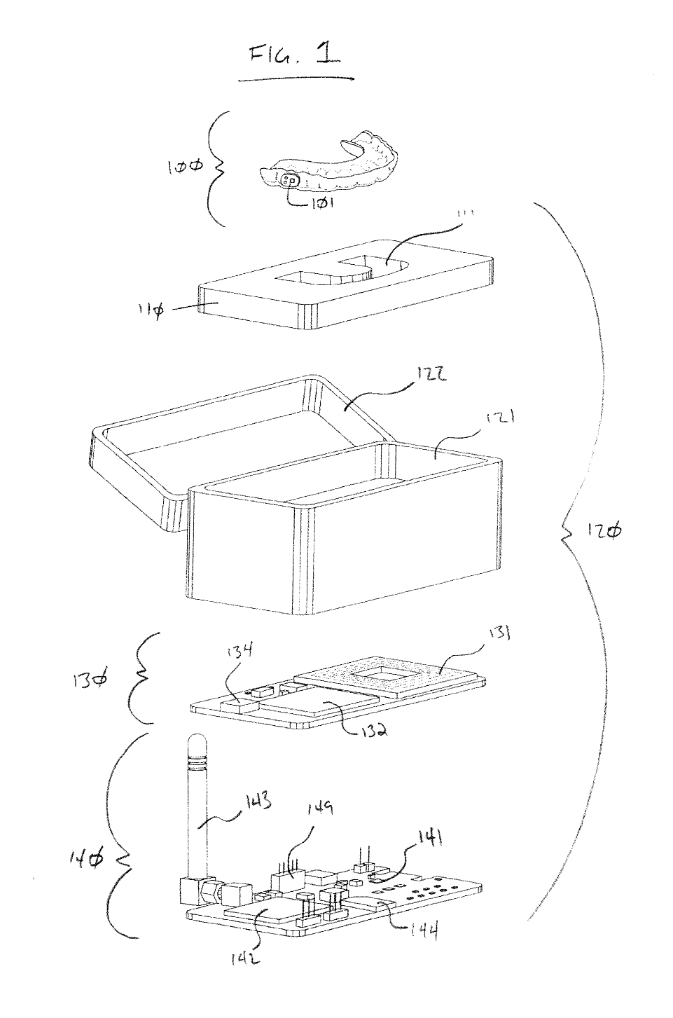

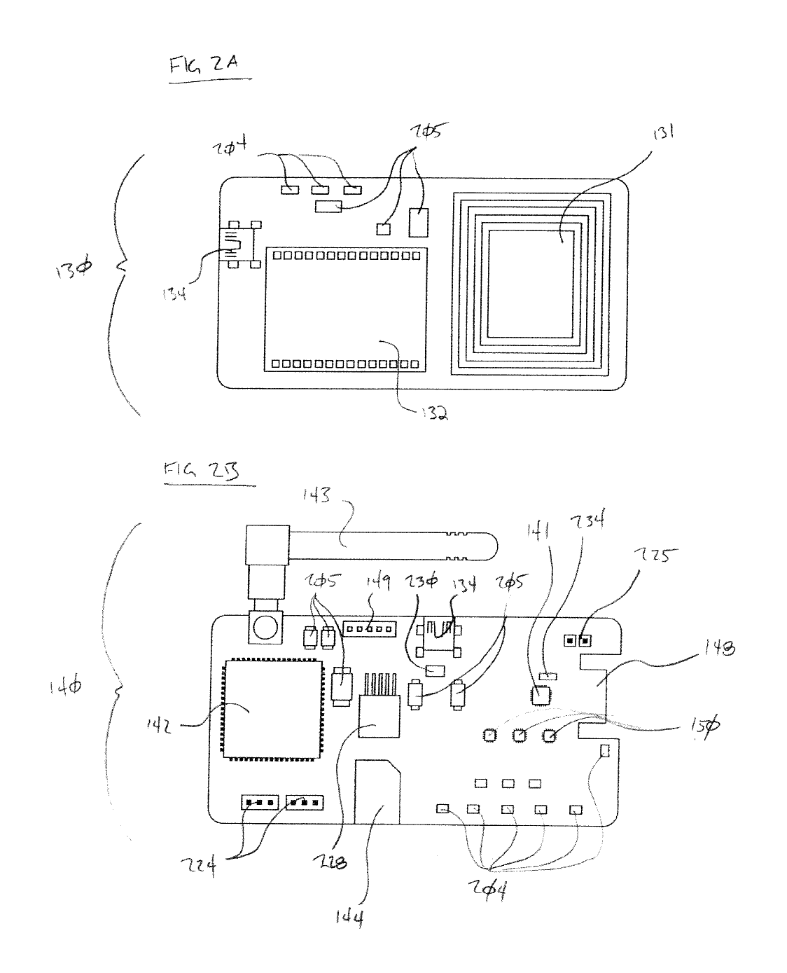

FIG. 1 illustrates an exploded perspective view of an embodiment of a reader box 120. The reader box comprises an oral appliance placement interposer 110, a compartment 121, a top printed circuit board (PCB) 130, and a bottom PCB 140. An oral appliance 100, containing embedded electronics 101, is placed inside the compartment 121 of the reader box 120. A protective lid 122 may close to protect the compartment 121 contents. An oral appliance placement interposer 110 contains the negative form of a patient's teeth 111 allowing for exact positioning of the oral appliance 100 within the reader box 120 with respect to the RFID antenna 131. The top PCB 130 comprises an RFID module 132, an RFID antenna 131, and RFID debugging port 134. Additional top circuit board 130 components which are not clearly visible in the FIG. 1 exploded perspective view are indicated on the same circuit board in a top-down view in FIG. 2A. When in the oral appliance 100 is seated in compartment 121, the RFID module 132 and RFID antenna 131 detect the presence of the oral appliance 100, download its data, update any parameters, and recharge its battery. The bottom PCB 140 contains the remaining electronics to implement the reader box 120. The downloaded data is optionally processed by a microprocessor 141 and saved in a storage device (located internally to the microprocessor 141). The cellular module 142, cellular antenna 143, and SIM card 144 provide connectivity to the cellular network. The cellular network enables issuing notifications and a direct connection to a server remote computer. Additional bottom circuit board 140 components which are not clearly visible in the FIG. 1 exploded perspective view are indicated on the same circuit board in a top-down view 140 in FIG. 2B. The top and bottom PCBS, 130 and 140 respectively, make up an oral appliance-reader communication module.

FIG. 2A illustrates a top-down perspective of an embodiment of a top PCB 130 implementation for an oral appliance-reader communication module. An RFID antenna 131 is printed directly onto the top PCB 130. A commercial RFID module 132 directly interfaces with the RFID antenna 131. Indicators such as LEDs 204 may be placed directly on the PCB. Power conditioning bypass capacitors 205, are placed as required by the application. A debug USB port 134 may provide a communication link between an external computer and the RFID of top PCB 130. The same debug USB port 134 may provide power to the RFID of top PCB 130.

FIG. 2B illustrates a top-down perspective of an embodiment of a bottom PCB 140 implementation for an oral appliance-reader communication module. A commercial cellular module 142 with antenna 143 and SIM card 144 provide connectivity to all resources that may be enabled via a cellular connection including, but not limited to, data, voice, and SMS. The cellular debugging ports 224 enable a connected remote computer to directly send and receive commands to the cellular module 142 as well as perform other maintenance such as cellular module 142 firmware upgrades. The cellular module power jumper 225 enables manual powering on or off of the cellular module 142. A system-on-chip (SoC) microprocessor 141 issues commands to the cellular module 142 and the RFID module 132. The microprocessor 141 may also receive commands from various input ports including a USB port 134 and debugger port 148. Debugger port 148 may be used to update the firmware of the microprocessor 141. A crystal oscillator 234 provides an accurate time base for the reader. LED indicators 204 may be used as a primary output device. Output may also be pushed through the USB port 134 and/or debugger port 148. USB port 134 additionally serves as the power port for both the bottom PCB 140 and top PCB 130. Numerous power conditioning circuits are required. A high-power low drop-out regulator 228 is required to provide a constant voltage source with a high amperage rating to the cellular module 142. Bypass capacitors of various sizes 205 are used to filter and condition the cellular voltage source. A low drop-out regulator 230 is used to provide a constant voltage source for the microprocessor 141. Bypass capacitors 205 are used to condition the microprocessor voltage source. Level-shifters 150 enable bi-directional communication between microprocessor 141 and all peripherals including RFID module 132, cellular module 142, USB port 134, and debugger port 148. Pin interface 149 connects the top PCB 130 in FIG. 2A to bottom PCB 140 in FIG. 2B.

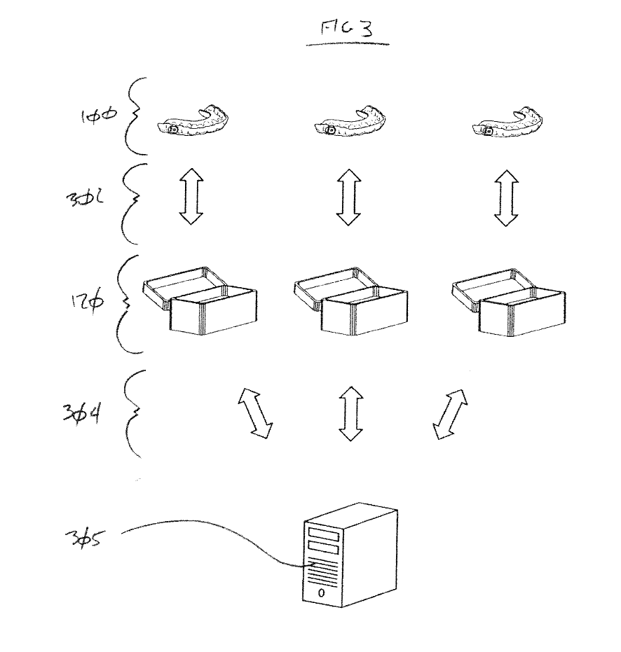

FIG. 3 illustrates an example embodiment of a Point-of-Care System (PCS) flow-diagram comprising three components: oral appliances 100, reader boxes 120, and a remote computer 305. Communication 302 between oral appliances 100 and reader boxes 120 occur when the oral appliances 100 are not in use and are physically placed within their respective reader boxes 120. The oral appliances 100 communicate with the reader boxes 120 via a connection 302 (e.g., an RFID connection). Connection 302 may also be used to recharge the oral appliance if necessary. The reader boxes 120 connect through a network 304 (e.g., a cellular network) to a remote computer 305. The network 304 may require authenticated credentials and encryption of data. The data stored in remote computer 305 may be accessed through an application interface (API). A user interface such as a website portal or program may be built upon an API for easy to use access.

In embodiments, the reader box 120 may be capable of wirelessly communicating via connection 302 with a hermetically sealed (or non-hermetically sealed) oral appliance 100 using wireless technology including, but not limited to, RFID, Near Field Communication (NFC), proprietary RF protocol, and/or infrared communication. A reader box 120, may communicate with the oral appliance 100 when placed in the compartment 121 of the reader box 120. The wireless communication may occur in a continuous fashion, for example, streaming the data from an oral appliance to the reader. Alternatively, the wireless communication may occur intermittently. In embodiments comprising an intermittent wireless communication mode, the oral appliance 100 may queue data in its memory storage for an interval and then upload the data as needed for each download request. The data may be uploaded or downloaded at any time and stored for an interval of any length. Intervals may be predetermined, preprogrammed, and/or may be random, occurring for only as long a time until download of the data is requested. In some embodiments, the same connection 302 may be used to wirelessly recharge the oral appliance 100 batteries. For example, RFID technology may be used to both communicate and recharge the oral appliance wirelessly. For example, specific RFID modules 132 may repurpose the RFID antenna 131 to continuously capture electromagnetic radiation, rectify the signal, and provide a constant DC charging current. In general, any RF antenna 131 including RFID, Bluetooth®, or Wi-Fi antennas may be repurposed to capture or harvest radiation and inductively trickle charge a battery.

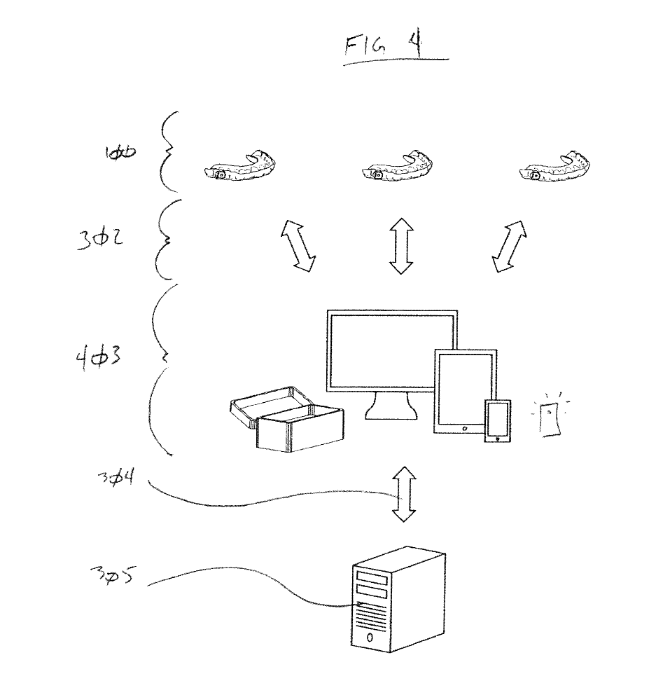

FIG. 4 illustrates an example embodiment of multiple PCS flow-diagram providing real-time data while actively in use. One or more oral appliances 100 may concurrently connect to a single or multiple readers 403 (e.g., a computer, tablet, smart device, reader box, relay transmitter, and the like). Data passed through connection 302 (e.g., a Low-Energy Bluetooth® connection) is transmitted while the oral appliances 100 are in the mouth. Connection 302 may transmit a live stream of data, notifications, or alerts. The reader 403 may provide a direct human interface for input parameters for the oral appliances 100 and visualization and storage of data. Alternatively, reader 403 may simply forward data directly to the remote server 305. Reader 403 may take the foam of the reader box 120, or may be a computer, tablet, smart device, reader box, relay transmitter, and the like. The network 304 connects the reader to the remote computer 305 through a network connection (e.g., cellular connection). The data stored in remote computer 305 may be may be accessed through an API. A user interface such as a website portal or program may be built upon an API for easy to use access.

As described above, embodiments comprise a PCS. Embodiments of the PCS comprise a reader 403. Embodiments of the reader may comprise a microprocessor 141, a data storage device (optionally located within microprocessor 141), and in some applications, a compartment 121 for storage of the oral appliance 100. Embodiments comprise a reader 403 that may transmit to and receive information from an oral appliance 100. Embodiments of the PCS comprise support hardware for the reader 403 including, but not limited to, high power regulator 228, low power regulator 230, level shifters 150, and/or crystal oscillators 234. The PCS may be used with a variety of oral appliances including night mouth guards, sports mouth guards, obstructive sleep apnea (OSA) oral appliances, orthodontic retainers, occlusal splints, complete removable dental prosthetics, and partial removable dental prosthetics.

In embodiments, the reader 403 may communicate with an oral appliance 100 through a bi-directional communication medium (e.g., connection 302). The reader 403 may include hardware for transmitting information to and from a remote computer 305 including, but not limited to, a cloud server, a personal computer, and/or a smart phone. Certain reader 403 embodiments may comprise a built-in rechargeable battery. Without limitation, reader 403 embodiments may comprise output devices including LEDs 204, monitor screens, external devices, and/or paired remote computers 305. Without limitation, other embodiments may comprise input devices including tactile buttons, key boards, touch screens, external devices, and/or remote computers 305 capable of configuring any element of the PCS.

In embodiments, the reader 403 may be capable of wirelessly communicating via connection 302 with a hermetically sealed (or non-hermetically sealed) oral appliance 100 using wireless technology including, but not limited to, RFID, Wi-Fi, Bluetooth®, Zigbee®, proprietary RF protocol, and/or infrared communication. In embodiments comprising a reader box 120, the reader 403 may communicate with the oral appliance 100 when placed in the compartment 121 of the reader box 120. In alternative embodiments, not comprising a reader box 120, the reader 403 may communicate with the oral appliance 100 through live stream/alerts that communicate via connection 302 while in use with a reader peripheral. The wireless communication may occur in a continuous fashion, for example, streaming the data from an oral appliance to the reader. As an example, a reader 403 (e.g., smart phone) may be paired with an oral appliance 100 through a Low Energy BlueTooth® connection 302 to bi-directionally stream voice data between an oral appliance 100 and the smart phone reader 403. A speaker placed in oral appliance 100 may transmit sound through the mandible or maxilla via the dentition, dental implants, or other oral or prosthetic structures, to the ear, and a microphone in the oral appliance 100 may captures and send the voice data to the smart phone reader 403. Alternatively, the wireless communication may occur intermittently. In embodiments comprising an intermittent wireless communication mode, the oral appliance 100 may queue data in its memory storage for an interval and then upload the data as needed for each download request. The data may be uploaded or downloaded at any time and stored for an interval of any length. Intervals may be predetermined, preprogrammed, and/or may be random, occurring for only as long a time until download of the data is requested. In some embodiments, subsets of data may be transmitted, providing early indication of interesting events in the data set. For example, in a concussion monitoring situation (e.g. a contact sport such as a football game), a red level alert (indicating a strong motion concussive hit) may be transmitted from the oral appliance 100 to the reader 403 via a connection 302 (e.g., RF medium) if a player receives a concussive level blow to the head. Reader 403 may relay this information to the coach or monitoring medical personnel and the player may be removed from further contact situations so that a full download and analysis of the oral appliance 100 data may be completed. After a full download, the oral appliance memory may then be cleared to log new data.

Some reader embodiments may comprise hardware for direct input and output interaction. Direct input methods may comprise tactile buttons, keyboards, touch screens, and the like. Direct output methods may comprise LED indicators 204, screens, speakers, and the like. Output information may include, but is not limited to, cellular status information, cellular signal strength, a “proper mounting in the reader” indicator, a charge indicator, a battery level indicator, alarm clock sounds, time, health scores, concussion summaries, oral appliance 100 microphone volume, oral appliance 100 speaker volume, usage history, pricing information, subscription information, and social network information. Input information may include, but is not limited to, configuration and parameter modification, sampling rate while docked, sampling rate while deployed, thermocouple offset and gain calibration settings, accelerometer offset and gain calibration settings, accelerometer dynamic range settings, accelerometer sampling rate, actigraphy threshold level, tilt sensor axis enable, oral appliance microphone volume, oral appliance speaker volume, and the like. When used in conjunction with a Companion Sensor (examples of companion sensors are described in U.S. patent application Ser. No. 13/934,432), burst number, burst interval, and other companion sensor settings may be included.

In embodiments, the reader 403 may adjust the parameters of the oral appliance 100. The adjustable oral appliance 100 parameters may be mechanical, electrical, and/or software in nature. An example of making an adjustment comprises the reader 403 downloading temperature data from the oral appliance 100, and in conjunction with a precision thermometer, the reader 403 adjusts the temperature offset calibration in the oral appliance 100. In reader box 120 embodiments, the reader box 120 may comprise an actuator that can turn a screw in an oral appliance 100 based on the oral appliance's data or a preprogrammed regimen. For example, many OSA appliances and retainers have mechanical worm screw adjustors. In embodiments, the reader 403 may be pre-programmed with a set of instructions, such as those provided by a clinician, to automatically adjust oral appliance 100 parameters by a given schedule to follow a prescribed regimen. In alternative embodiments, the reader 403 may adopt new parameters upon (independent or dependent) analysis of the oral appliance's 100 recorded data to reduce or mitigate errors registering in a feedback system. In alternative embodiments, the reader 403 may indicate to the patient or to the clinician when and to what degree to make manual changes to the oral appliance 100. The oral appliance 100 parameters may be adjustable by a remote computer 305 or a user through a portal accessed via the reader's network 304. Example parameters which the reader 403 may update include, but are not limited to, sensor sampling rates, sensor sensitivity, the time base, and remote disable.

In embodiments, the PCS may enable continuous monitoring. Continuous monitoring may provide better long-term care for chronic conditions while also continuously monitoring important metrics for indicators of potential problems. In embodiments comprising a server remote computer 305, the patient and clinician may access data and manage parameters easily and transparently via a portal or with a third party through an API. The notification system may enable the reader 403 and/or remote computer 305 to notify interested parties of certain events and even trigger a visit between the patient and clinician. For example, in a sports medicine setting, in situ head acceleration monitoring may transmit notifications of the frequency and severity of the head impacts of athletes wearing an oral appliance during a game. For example, three (or any number of) accelerometers may be placed in a configuration (e.g., a triangular configuration) in the oral appliance 100 in such a way that linear X, Y and Z accelerations as well as yaw, pitch, and roll accelerations are measured. Rotational accelerations are measured by using multiple accelerometers placed in a known configuration, and calculating the torque between any two linear accelerometers. Without being limited by theory, embodiments comprising many linear accelerometers may derive yaw, pitch roll without a gyro For example, with two accelerometers you can find two of the three rotational accelerations and rotational velocities, therefore comprising a total of five degrees of information. With at least three accelerometers all six degrees of motion may be calculated. In embodiments, accelerometers may be used in place of gyros, to provide low energy solutions. The acceleration magnitude and direction are compared against a concussion severity index (SI) rubric. Such a strong motion notification system may take the form of a green/amber/red alert indicating levels on the concussion SI. With such information, a coach may pull a player from a game and trigger a visit to a specialist. In another example, in fatigue and sleep monitoring embodiments for commercial motor vehicle drivers, after every nightly use, the oral appliance may be placed in a reader box 120 and the compliance usage and assessment data (e.g., actigraphy data) may then be downloaded, examples of which can be found in U.S. Provisional Patent Application 61/892,232 filed on Oct. 17, 2013 which is incorporated herein by reference). Based on the recorded sleep data, a sleep quality index notification on a scale from 0 to 100 may be transmitted to the patient or interested party to indicate the level of compliance during the current night or during the aggregate of many previous nights. This information may trigger the patient to take corrective action to meet corporate or regulatory guidelines. The system may be scalable in that it may simultaneously monitor or treat numerous patients with minimal incremental overhead. The oral appliance 100 battery may be rechargeable, eliminating waste associated with depleted monitors as well as reducing the need to replace oral appliances with new electronics. The reader's 403 oral appliance placement interposer 110 may allow it to overcome the many obstacles associated with the placement of unique and custom appliances in the reader 403. The reader 403 may additionally self-diagnose itself and the oral appliance 100 to notify the original equipment manufacturer (OEM), the service provider or a clinician if the hardware is in need of repair or replacement.

Additional reader box 120 embodiments may comprise positioning the oral appliance 100 in the reader box 120 to enable communication between the oral appliance 100 and the reader box 120 and to recharge the oral appliance 100. An example embodiment of this method includes a removable oral appliance placement interposer 110 which fits within the reader box 120. This may be implemented as a surface positive of the intaglio surface of the oral appliance 100. Alternatively, the orienting surface could be formed as a surface negative 111 of the cameo surface of the oral appliance 100. An additional embodiment may include creating a positive of the negative orienting surface based on the oral structures (other than the teeth) and the unique shape and characteristics of the oral appliance 100 itself. The orienting surface may be standardized to accommodate a pre-determined arch size or to the form of an oral appliance 100 or it may be custom fitted for each individual oral appliance 100. In some embodiments, the custom negative or positive orienting surfaces may be generated using 3D scanning tools, 3D computer aided design (CAD) software, and/or SLA manufacturing. Embodiments of stock negative or positive orienting surfaces may be generated using SLA manufacturing, vacuum formable, thermo formable, and/or injection molded plastic. In example embodiments, the custom or stock orienting surface may be inserted into the reader box 120 in order to key the precise location of the oral appliance 100 within the reader box 120. Keying the reader box 120 with an orienting surface may ensure a repeatable and precise placement of the oral appliance 100 within the reader box 120. An additional embodiment may include the use of magnetics between the oral appliance 100 and reader box 120 to repeatedly and precisely place the oral appliance 100 within the reader box 120. A magnet placed within each component will force the oral appliance 100 and the reader box 120 to pair in a predesigned configuration.

Reader embodiments may comprise power options such as a built-in rechargeable battery and the ability to connect to a variety of power sources. Some embodiments may comprise a built in battery that enables the reader to recharge and continue operating even while not connected to an external power source. Some embodiments comprise plug options which may enable the reader to function in a variety of environments to address different market segments. Example plug options may include any USB® style plug (e.g., USB port 134), a traditional wall wart plug, or the ability to plug into a vehicle outlet. In embodiments comprising plugs capable of data transfer, the reader 403 may be capable of communicating with the connected device.

Some reader box 120 embodiments may include sanitation and germicide options to sterilize oral appliances 100 between uses. In one embodiment, an ultraviolet light may be used to disinfect the oral appliance 100 when inserted inside a reader box 120. The ultraviolet light may be triggered to illuminate when the oral appliance 100 has been inserted or removed from the reader box 120.

The reader box 120 may comprise multiple compartments 121 for storage of oral appliances 100. Oral appliances 100 may sometimes comprise more than one piece; thus the reader box 120 may comprise a compartment 121 for each piece. In optional embodiments, the compartments 121 may be keyed. In embodiments, each storage compartment 121 may detect an inserted oral appliance 100, may communicate 302 bi-directionally with the oral appliance 100, and may recharge the oral appliance 100. RF and optical methods of detection, communication, and/or charging are examples that may be used in compartments 121.

In embodiments, the reader box 120 may be any shape. In example embodiments, the reader box 120 may be rectangular with rounded edges. In embodiments, the reader box 120 may comprise a lid 122 that protects the contents of the reader box 120. The reader box 120 size may be any such size necessary to contain an oral appliance 100. The reader box 120 may provide personalization by indicating the patient's name or customer's name formed in plastic directly on the reader box 120. Other information may be foamed in plastic as well. The reader box 120 color may be customizable by the user or customer. Reader box 120 personalization options may be implemented using 3D printing technology, thermal forming, or injection molded plastic technologies. The electronics and hardware are housed in an internal compartment. Embodiments may comprise one or more windows or openings for input and output devices.

Reader 403 embodiments may comprise materials specifically optimized for certain or multiple purposes. For example, the reader 403 material may be specifically optimized to allow RF energy through with little loss, such as might be radiated by a cellular, RFID, Bluetooth®, or Wi-Fi antenna. Additionally, the entirety or parts of the reader 403 may be designed with a material indicated by the Food and Drug Administration as bio-compatible or food safe grade. Without limitation, examples of biocompatible materials that may be used include polypropylene, polystyrene, poly methyl methacrylate, polycarbonates, acrylonitrile butadiene styrene, high-impact polystyrene, PolyJet® photopolymer, and combinations thereof.

Additional embodiments of the PCS may further comprise one or more remote computers 305. Remote computers 305 are servers or computers used to facilitate the deployment of the PCS. Remote computers 305 may be used to overcome size, cost, processing power, memory storage capacity, battery limits, input and output limitations, and/or communication limitations of a standalone reader 403.

Certain reader 403 embodiments may include a method of transmitting and receiving information to a remote computer 305. Remote computers 305 may include personal computers, smart phones, cloud servers, and/or other remote devices configured to communicate with a reader. The reader 403 may communicate with the remote computer 305 through a wired or wireless network, examples of which include, but are not limited to, debug busses, UART, RS-232, USB, Ethernet, Zigbee®, Wi-Fi, Bluetooth®, cellular networks, and/or any other network as would occur to one skilled in the art. Proxy devices such as routers, smart phones, gateways, and/or cellular base stations may be used to facilitate communication between the reader and a remote computer. When the reader 403 is connected to a remote computer 305, numerous features may be enabled which, in some embodiments, may not be possible with the reader 403 alone. In some embodiments, the remote computer 305 may function as a debugger (e.g., capable of downloading and installing new firmware into the reader's memory). In some embodiments, the remote computer 305 may comprise a method for performing diagnostic and testing routines on the reader 403. The remote computer 305 may serve as a general purpose output port for the reader 403, enabling logged data and status outputs to be visualized. The remote computer 305 may serve as a general purpose input port for the reader 403, enabling the remote computer 305 to reconfigure the reader's 403 settings. The remote computer 305 may comprise a clock for time keeping and data recording purposes. Additionally, the remote computer 305 may push notifications directly to patients, clinicians, or other interested parties. The data at-rest in the remote computer 305 and the data in-flight on the network 304 may be fully encrypted and compliant with any regulations, such as government mandated medical privacy regulations. An example embodiment comprises a reader 403 with a cellular module 142 connected wirelessly through a 2G network 304 to a cloud server remote computer 305. The reader 403 may download the time directly from the cellular network 304. A notification system may be implemented via SMS or voicemail services. The cellular network's 304 data services may provide connection to the server remote computer 305 through the internet. The server remote computer 305 may be hosted on a secure connection 302 or secure connection 302, protected with username password authentication as well as encryption to comply with regulatory requirements. An additional example embodiment comprises a reader 403 paired with a smart-phone remote computer 305 though a paired Bluetooth® connection 302 or connection 302. The smart-phone remote computer 305 may be used to connect to, download data, and configure the reader's 403 parameters.

The remote computer 305 interface may vary depending on the specific device and application. In embodiments where a smart phone serves as the remote computer 305, a smart phone application may be used as the interface. In embodiments where a personal computer serves as the remote computer 305, a program or executable may be used as the interface. In embodiments where a server serves as the remote computer 305, a website or portal may be used as the interface. Alternatively, a server remote computer 305 may provide a machine interface through an API.

An example of a PCS may comprise a reader 403 and a remote computer 305. The reader 403 connects directly to an oral appliance 100. In this example embodiment, the remote computer 305 cannot directly connect to the oral appliance 100. In practice, such as in a football game, the reader 403 may directly pair with the oral appliance 100 (e.g., a mouth guard) and send real-time notifications through a Bluetooth® connection 302. Additionally, should the oral appliance 100 also comprise RFID transmittal capability, the player may also place the oral appliance in a reader box 120 wherein the reader box 120 may download data and recharge the battery of the oral appliance 100 through an RFID connection 302. The same reader 403 previously used to send and receive real-time notifications may then be used to access a server's 305 portal and see the full log of data downloaded by the reader box 120.

A remote computer 305 may comprise a secure and encrypted connection for the reader 403 over a network 304. In example embodiments, the remote computer 305 may be a cloud server. The server application may comprise a database and an API. A portal may be built on top of the server's API. A portal may be any user interface used to access the server's database. Common portals include websites and smart device applications. Without limitation, API functions include managing patient profiles, hardware configuration profiles, associations between patients and hardware, billing, user roles, accessing data, modifying reader parameters, modifying oral appliance parameters, and other tasks as required by the application.

In embodiments, the remote computer 305 may process the oral appliance 100 data and generate numerous useful outputs. The remote computer 305 may store a log of all data and process the entirety of the data or a subset of the data using any method of analysis as would occur to one skilled in the art. For example, temperature and actigraphy data may be processed together to determine sleep compliance and sleep quality during a specified time range. In embodiments the oral appliance 100 may comprise additional sensor types to measure different metrics. Without limitation, examples of sensors include accelerometers, gyros, inertial measurement sensor (IMU), pulse oximeter, actigraphy sensor, potentiometry sensor, coulometry sensor, voltammetry sensor, amperometry sensor, capnography sensor, compliance monitor sensor, or combinations thereof. Compliance algorithms and methods may include those as indicated in a Companion Sensor system (examples of companion sensors and companion sensor systems are described in U.S. patent application Ser. No. 13/934,432, the entire disclosure of which is incorporated herein by reference). Actigraphy methods may include proprietary or known algorithms for associating head acceleration data to sleep quality. The resulting processed data may provide useful indicators to the users, customers, or any interested party.

In embodiments, a health interrupt system may be implemented to induce a patient to visit a clinician based on complex health metrics. A health-interrupt system may be implemented as the output of an algorithm, which analyzes a patient's bioinformatic data to discover useful trends and signals buried in the data's noise. Statistical models may find significant correlations between different vital signs as an indicator of a severe condition. For example, an elderly patient with an immune disorder may require constant monitoring to catch signs of an infection requiring immediate attention. An oral appliance 100 may be used to continuously measure temperature and other vital signs. The life-sign data is processed, and an early warning of an infection health-interrupt may trigger the patient to visit a clinician, or may notify a care-taker to check on the patient. As another example, in large data sets, small useful correlations between measured data, a patient's health history, or genetic profile may indicate health implications that would otherwise be practically impossible to discover. For example, by stacking the data, or combing the datasets such that the useful correlations add, while uninteresting correlations cancel, the useful and actionable health-interrupt metrics may be discovered and coded into the reader or remote computer's program. The remote computer may comprise a method to generate and transmit health scores based on the logged data. For example, in a compliance monitoring embodiment, health scores may be used to encourage patients to minimize non-compliant behavior. Health scores may also be integrated into social media websites to share information for entertainment or health purposes. In embodiments, the remote computer may generate one or more health reports as medical records. The methods of processing data and generated outputs are not limited to the previously described methods and may include others as applicable.

The remote computer 305 may change the parameters of the reader 403. In embodiments where the reader 403 is connected to a remote computer 305, a firmware upgrade may be downloaded to correct or update the reader's 403 programming. The remote computer 305 may also comprise methods for updating its own parameters. These parameters may be updated by a remote computer's 305 website, an application, or an executable. The remote computer 305 may receive notifications such as SMS messages or voicemails indicating changes in functions or parameters. Example parameters may include remote enable/disable, URLs used to post data and retrieve updates, polling rates, sensitivity settings, and any other parameterized settings.

Embodiments may include, but are not limited to uses in a sports-medicine, sleep monitoring, monitoring the effectiveness of treatments, non-invasive monitoring during surgical, clinical, or hospital settings, elderly vital sign monitoring, and for general entertainment.

It should be understood that the compositions and methods are described in terms of “comprising,” “containing,” or “including” various components or steps, the compositions and methods can also “consist essentially of” or “consist of” the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present embodiments are well adapted to attain the ends and advantages mentioned as well as those that are inherent therein. The particular embodiments disclosed above are illustrative only, and they may be modified and practiced in different but equivalent manners apparent to those skilled in the art having the benefit of the teachings herein. Although individual embodiments are discussed, all combinations of all those embodiments are covered by the disclosure. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of those embodiments. If there is any conflict in the usages of a word or term in this specification and one or more patent(s) or other documents that may be incorporated herein by reference, the definitions that are consistent with this specification should be adopted.

What is claimed is:

1. A method for obtaining data from an oral appliance, the method comprising:
placing an oral appliance comprising embedded electronics in an oral cavity, wherein the embedded electronics collect data;
transmitting the data to a reader, wherein the reader comprises a computer, tablet computer, smart device, reader box, relay transmitter, or a combination thereof.

2. The method of claim 1 further comprising the reader and the oral appliance being capable of bi-directional communication.

3. The method of claim 2 wherein the reader and the oral appliance communicate in a bi-directional method comprising at least one bi-directional communication method selected from the group consisting of RFID, Wi-Fi, Bluetooth, Bluetooth Low-Energy, ANT, Zigbee, infrared communication, Sub-Ghz RF, proprietary RF standard, body area network, and any combination thereof.

4. The method of claim 1 wherein the oral appliance is a mouth guard, obstructive sleep apnea oral appliance, orthodontic retainer, occlusal splint, a complete removable dental prosthetic, or a partial removable dental prosthetic.

5. The method of claim 1 wherein the reader communicates with a remote computer and wherein the remote computer communicates with the reader.

6. The method of claim 5 wherein the remote computer comprises a smart-phone and an interface, a personal computer and an interface, or a server and an interface.

7. The method of claim 5 further comprising the step of sending the collected data to the remote computer from the reader.

8. The method of claim 5 wherein the reader and the remote computer communicate in a bi-directional method comprising at least one bi-directional communication method selected from the group consisting of RFID, Wi-Fi, Bluetooth, Zigbee, infrared communication, cellular network, wired Ethernet, USB, and any combination thereof.

9. The method of claim 5 further comprising the step of transmitting user input adjustments to the reader from the remote computer.

10. The method of claim 9 further comprising the step of adjusing the oral appliance in response to the user input adjustments sent to the reader from the remote computer.

11. The method of claim 1, wherein the embedded electronics further comprises an accelerometer, gyro, pulse oximeter, inertial measurement sensor, actigraphy sensor, potentiometry sensor, coulometry sensor, voltammetry sensor, amperometry sensor, capnography sensor, compliance monitor sensor, or combinations thereof.

12. The method of claim 1 wherein the embedded electronics further comprise a speaker and a microphone, and wherein the reader and the oral appliance bi-directionally stream sound and/or voice data between each other.

13. A system for obtaining data from an oral appliance, the system comprising:
an oral appliance;
a reader; wherein the reader is configured to communicate with the oral appliance, and wherein the reader is configured to download data from the oral appliance;
a remote computer, wherein the remote computer is configured to receive transmitted data from the reader, and wherein the reader and the remote computer are configured to bi-directionally communicate; and
a portal, wherein the portal is configured to display the data transmitted to the remote computer.

14. The system of claim 13 wherein the oral appliance is a mouth guard, obstructive sleep apnea oral appliance, orthodontic retainer, occlusal splint, a complete removable dental prosthetic, or a partial removable dental prosthetic.

15. The system of claim 13 wherein the reader and the oral appliance communicates in a bi-directional method comprising at least one bi-directional communication method selected from the group consisting of RFID, Wi-Fi, Bluetooth, Bluetooth Low-Energy, ANT, Zigbee, infrared communication, Sub-Ghz RF, proprietary RF standard, body area network, and any combination thereof.

16. The system of claim 13 wherein the reader and the remote computer communicate in a bi-directional method comprising at least one bi-directional communication method selected from the group consisting of RFID, Wi-Fi, Bluetooth, Zigbee, infrared communication, Sub-Ghz RF, proprietary RF standard, cellular network, wired Ethernet, USB, and any combination thereof.

17. The system of claim 13 wherein the remote computer comprises a smart-phone and an interface, a tablet and an interface, a personal computer and an interface, or a server and an interface.

18. The system of claim 13 wherein the remote computer is configured to send user input adjustments to the reader.

19. The system of claim 18 wherein the reader is configured to adjust the oral appliance in accordance with the user input adjustments sent to the reader from the remote computer.

20. The system of claim 13 wherein the reader is configured to recharge the oral appliance.
